SEQUENCE LISTING (24 sequences)

SEQ ID NO: 1 - DNA, 1299 bp, Homo sapiens
  CDS (1)..(1299)
gtggaaacagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacggaaccaggtgctgaccctgtatctgtggatacggcaggagtggacagatgcctacctacgatgggaccccaatgcctatggtggcctggatgccatccgcatccccagcagtcttgtgtggcggccagacatcgtactctataacaaagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctgcctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcatgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctg

SEQ ID NO: 2 - protein, 433 aa, Homo Sapiens
VETECLGAEGRLALKLFRDLFANYTSALRPVADTDQTLNVTLEVTLSQIIDMDERNQVLTLYLWIRQEWTDAYLRWDPNAYGGLDAIRIPSSLVWRPDIVLYNKADAQPPGSASTNVVLRHDGAVRWDAPAITRSSCRVDVAAFPFDAQHCGLTFGSWTHGGHQLDVRPRGAAASLADFVENVEWRVLGMPARRRVLTYGCCSEPYPDVTFTLLLRRRAAAYVCNLLLPCVLISLLAPLAFHLPADSGEKVSLGVTVLLALTVFQLLLAESMPPAESVPLIGKYYMATMTMVTFSTALTILIMNLHYCGPSVRPVPAWARALLLGHLARGLCVRERGEPCGQSRPPELSPSPQSPEGGAGPPAGPCHEPRCLCRQEALLHHVATIANTFRSHRAAQRCHEDWKRLARVMDRFFLAIFFSMALVMSLLVLVQAL

SEQ ID NO: 3 - DNA, 1501 bp, Homo sapiens
  CDS (34)..(1386)
gagacctgcccccgctcttgcagtgccagggccatggggctccggagccaccacctcagcctgggccttctgcttctgtttctactccctgcagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacggaaccaggtgctgaccctgtatctgtggatacggcaggagtggacagatgcctacctacgatgggaccccaatgcctatggtggcctggatgccatccgcatccccagcagtcttgtgtggcggccagacatcgtactctataacaaagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctgcctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcatgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctgtgagggctgggactaagtcatctagagggcccttcgaaggtaagcctatccctaaccctctcctcggtctcgattctacgcgtaccggtcatcatcaccatcaccattgagtttaaac

SEQ ID NO: 4 - protein, 450 aa, Homo sapiens
MGLRSHHLSLGLLLLFLLPAECLGAEGRLALKLFRDLFANYTSALRPVADTDQTLNVTLEVTLSQIIDMDERNQVLTLYLWIRQEWTDAYLRWDPNAYGGLDAIRIPSSLVWRPDIVLYNKADAQPPGSASTNVVLRHDGAVRWDAPAITRSSCRVDVAAFPFDAQHCGLTFGSWTHGGHQLDVRPRGAAASLADFVENVEWRVLGMPARRRVLTYGCCSEPYPDVTFTLLLRRRAAAYVCNLLLPCVLISLLAPLAFHLPADSGEKVSLGVTVLLALTVFQLLLAESMPPAESVPLIGKYYMATMTMVTFSTALTILIMNLHYCGPSVRPVPAWARALLLGHLARGLCVRERGEPCGQSRPPELSPSPQSPEGGAGPPAGPCHEPRCLCRQEALLHHVATIANTFRSHRAAQRCHEDWKRLARVMDRFFLAIFFSMALVMSLLVLVQAL

SEQ ID NO: 5 - protein, 447 aa, Rattus sp.
MGTRSHYLDLGFLLLLFLPAECLGAEGRLAHKLFRDLFANYTSALRPVADTDQTLNVTLEVTLSQIIDMDERNQVLTLYLWIRQEWTDAYLHWDPKAYGDLDAIRIPSRLVWRPDIVLYNKADTQPPASASTNVVVRHDGAVRWDAPAITRSSCRVDVSAFPFDAQRCGLTFGSWTHGGHQLDVRPRGTSASLADFVENVEWRVLGMPARRRVLTYGCCSEPYPDVTFTLLLRRRAAAYVCNLLLPCVFISLLAPLAFHLPADSGEKVSLGVTVLLALTVFQLILAESMPPAESVPLIGKYYMATMTMVTFSTALTILIMNLHYCGPNAHPVPAWARVLLLGHLAKGLCVRERGEPCGQSKPLESAPSLQPPPASPAGPCHEPRCLCHQEALLHHIASIASTFRSHRAAQRRHEDWKRLARVMDRFFLGIFFCMALVMSLIVLVQAL

SEQ ID NO: 6 - protein, 452 aa, Gallus domesticus
MGSAPLPACLLALSLAGTVLAPGCGAAQGRLAHKLLHDLFANYSSALRPAEDTERALNVTLQVTLSQIIDMDERNQVLTSYLWVRQAWLDAHLAWDKDAYGGIDSIRIPSSYVWRPDIVLYNNADERFGGSMETNVVLRSDGHIMWDSPAITKSSCKVDVSYFPFDGQQCRLTFGSWTYNGNQIDLRNLLDTGDLTDFVENVEWEVLGMPATRNVVTYGCCSEPYPDVTYTLLLRRRASFYIFNLLLPCVMISFLAPLGFYLPADSGEKVSLGVTVLLALTVFQLLVAESMPPSESVPLIGKYYIATMTMITASTALTIFIMNIHHCGPGARPVPPWARRLILHHLARALCVCEVGESCGRPQREGTGGMGPRDPPGEGVEPGLCPRSRCLCHHHAVLSSVGYIAGVFRRHRTAQRRAAEWKKVAKVMDRFFMWVFFLMVFLMSVLVIGKAA

SEQ ID NO: 7 - protein, 479 aa, Homo sapiens
MNWSHSCISFCWIYFAASRLRAAETADGKYAQKLFNDLFEDYSNALRPVEDTDKVLNVTLQITLSQIKDMDERNQILTAYLWIRQIWHDAYLTWDRDQYDGLDSIRIPSDLVWRPDIVLYNKADDESSEPVNTNVVLRYDGLITWDAPAITKSSCVVDVTYFPFDNQQCNLTFGSWTYNGNQVDIFNALDSGDLSDFIEDVEWEVHGMPAVKNVISYGCCSEPYPDVTFTLLLKRRSSFYIVNLLIPCVLISFLAPLSFYLPAASGEKVSLGVTILLAMTVFQLMVAEIMPASENVPLIGKYYIATMALITASTALTIMVMNIHFCGAEARPVPHWARVVILKYMSRVLFVYDVGESCLSPHHSRERDHLTKVYSKLPESNLKAARNKDLSRKKDMNKRLKNDLGCQGKNPQEAESYCAQYKVLTRNIEYIAKCLKDHKATSSKGSEWKKVAKVIDRFFMWIFFIMVFVMTILIIARAD

SEQ ID NO: 8 - protein, 479 aa, Rattus sp.
MNRPHSCLSFCWMYFAASGIRAVETANGKYAQKLFSDLFEDYSSALRPVEDTDAVLNVTLQVTLSQIKDMDERNQILTAYLWIRQTWHDAYLTWDRDQYDRLDSIRIPSDLVWRPDIVLYNKADDESSEPVNTNVVLRYDGLITWDSPAITKSSCVVDVTYFPFDSQQCNLTFGSWTYNGNQVDIFNALDSGDLSDFIEDVEWEVHGMPAVKNVISYGCCSEPYPDVTFTLLLKRRSSFYIVNLLIPCVLISFLAPLSFYLPAASGEKVSLGVTILLAMTVFQLMVAEIMPASENVPLIGKYYIATMALITASTALTIMVMNIHFCGAEARPVPHWAKVVILKYMSRILFVYDVGESCLSPRHSQEPEQVTKVYSKLPESNLKTSRNKDLSRKKEVRKLLKNDLGYQGGIPQNTDSYCARYEALAKNIEYIAKCLKDHKATNSKGSEWKKVAKVIDRFFMWIFFAMVFVMTVLIIARAD

SEQ ID NO: 9 - DNA, 24 bp, Artificial Sequence
  Primer
ggaaaatgtgtgtgtcagtaaagc

SEQ ID NO: 10 - DNA, 20 bp, Artificial Sequence
  Primer
gaagtgttttcagagtgagg

SEQ ID NO: 11 - DNA, 17 bp, Artificial Sequence
  Primer
cagcaccaacgtggtcc

SEQ ID NO: 12 - DNA, 18 bp, Artificial Sequence
  Primer
ggcaccaactggatgtgc

SEQ ID NO: 13 - DNA, 19 bp, Artificial Sequence
  Primer
cacgttctccacgaagtcc

SEQ ID NO: 14 - DNA, 18 bp, Artificial Sequence
  Primer
cagccgtaggtgagcacg

SEQ ID NO: 15 - DNA, 20 bp, Artificial Sequence
  Primer
tggcaggcttttggacttcc

SEQ ID NO: 16 - DNA, 21 bp, Artificial Sequence
  Primer
tcttggcctttgtagagttcc

SEQ ID NO: 17 - DNA, 20 bp, Artificial Sequence
  Primer
tggcgccagaagatagtacc

SEQ ID NO: 18 - DNA, 19 bp, Artificial Sequence
  Primer
tcactccatggcccttacc

SEQ ID NO: 19 - DNA, 3834 bp, Homo sapiens
  CDS (59)..(140)
  CDS (516)..(816)
  CDS (2248)..(3238)
taatacgactcactatagggcggccgcggaggaaaatgtgtgtcagtaaagcctggggaagtgtgttttcagagtgagggagtgttccatcgcatcagaagttttgaagaaaccagctcgagatggagaagtggaaacaggtttgagagatactggagggggcagagcagtgggatttagaatccctgggtgaaagtctggactctcgtggcttatttgggcccctctagcatttgtggagaggcaggcagactccaggtccttgaaaaggggagggtggaggagaaatttgtcagcctggcgccagaagatagtaccagttcactccatggcccttacctcatgtgtccctgcaggcaggccagggaggaactagagccacagctagagcaagagaaggcagacaccaggaggacactcataaggacagggccccagccctgggagtggagggtgtgagcagaggccctgggactagggcctgggatggacaaccctccttactgaccctccagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacgggaccaggtgctgaccctgtatctgtggatacggcaggagtggacagatgcctacctacgatgggrccccaatgcctatggtggcctggatgccatccgcatccccagcagtcttgtgtggcggccagacatcgtactctataacaagtactgcctatctgggcccctcctctctcttacccctctctagacttgcccttagctgtgggggtgtagtgatcccctctccctaccacataacctggttgccacgctgccctggaagcttttccccaggacccttctaagctgccaagcactcagcccctccatggcacccccactttaggctatcccaggccagcccaggctgaacgtctcctcggaacctactgtgtggtccagggcagatgtctgaatcacaagggcctctctagggcacacttttagctctaagtctctcagggctcccccaagagcctgtctaagggtctctttcctccaggacatagccctctggaacactgctttatgtctccttgaccagttccgtgtctcccagccagcacatagctctgcatattttctctggggcccttctacaagttttgcagatgtcccccaagggaagtcactgtgtgtcccggagctacctctgggttctgcagaggcctttttatacatcctctggctacgtctgtgtcccttctgggcccttcaggcaccaccccttccaggcctcgaaaggcagcgggtctctctaggtgcactccaccctctgtgttgctttgttctgaaaacaagaatcaaattaacgaaaaaaaaacaagcacaagtttatttatttatttgagacacagtctcgctctgtcgcccaggctggagtgcagtggcgctatctcggctcactgcaagctccgcctcccgggttcacgcaattctcctgcctcaacctcccaaataactgggactgcaggcacccgccaccacgcccagctagttttttgtatttttagtagagacgaggtttcaccgtgttagccagggtggtctcgatctcctgacctcgtgatccgcccacctcggcctcccaaagtgctgggattacaggcgtgagccaccgcacccagccacaagcagaagtttattaatctgctgtacccatcatgggagaggccttagttcaaaagtatttctctctgaaggcagtgacttaggggccttgcttaaatagaaattcaagaaagagccagtaagttataaatagtggcaagacaaaggacagccacctttaaaaggcgggaaaacgtggaaagagggtaaaatctgtttccagattcctctggcacctactggtgccctttggataagcaagtgctgactccagcaaggaagggctgatgtcctgccatcaggccagcagacgctggggccaggtgctcccctgcgtcgtgagtgtctcgaacttaacgagcctcaatattctggggagaagttttggtttctttcagcccctgggggtctgccctgggctcccggcctccggggctgctcctcaggctggacagcctaggtgagccctgccccgcctgcccccagagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctacctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcacgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctgtgagggctgggactaagtcacagggatctgctgcagccacagctcctccagaaagggacagccacggccaagtggttgctggtctttgggccagccagtctctccccactgctcctaagatcctgagacacttgacttcacaatccacaagggagcactcattgtctacacaccctaactaaaggaagtccagagcctgccactcccctaattccaaaaaaaagaggaactctacaaaggccaagatcacagagtacagtcttggagggacagaattgtttgtgctgggtattggagctctcagtggggagcacatgggttataatgagaaactgaactgtactgctgcatttcctgtcttccttcctaggtggctgctttgcagggctttggctgttacctttccctgctgaggggctcagggaaaagggtcggggattctcagtcgagtttccagagcaggaggccctacagacatttggccccaaatccctgactcaataaagtaagcgtgtacctaaaaaaaaaaaaaaaaaactcgactctagattgcggccgcggtcatagctgtttcctgatctgaattcgtcgacaagctt

SEQ ID NO: 20 - protein, 457 aa, Homo sapiens
  SITE 101: Xaa=uncertain
KCVFRVRECSIASEVLKKPARDGEVETECLGAEGRLALKLFRDLFANYTSALRPVADTDQTLNVTLEVTLSQIIDMDERDQVLTLYLWIRQEWTDAYLRWXPNAYGGLDAIRIPSSLVWRPDIVLYNKADAQPPGSASTNVVLRHDGAVRWDAPAITRSSCRVDVAAFPFDAQHCGLTFGSWTHGGHQLDVRPRGAAASLADFVENVEWRVLGMPARRRVLTYGCCSEPYPDVTFTLLLRRRAAAYVCNLLLPCVLISLLAPLAFHLPADSGEKVSLGVTVLLALTVFQLLLAESMPPAESVPLIGKYYMATMTMVTFSTALTILITNLHYCGPSVRPVPAWARALLLGHLARGLCVRERGEPCGQSRPPELSPSPQSPEGGAGPPAGPCHEPRCLCRQEALLHHVATIANTFRSHRAAQRCHEDWKRLARVMDRFFLAIFFSMALVMSLLVLVQAL

SEQ ID NO: 21 - DNA, 25 bp, Artificial Sequence
  Description of Artificial Sequence Primer
cctccagggtcacattcagagtctg

SEQ ID NO: 22 - DNA, 20 bp, Artificial Sequence
  Description of Artificial Sequence Primer
cagcttgagagccagccggc

SEQ ID NO: 23 - DNA, 31 bp, Artificial Sequence
  Description of Artificial Sequence Primer
gcgaattcaggcctcacatccagagacctgc

SEQ ID NO: 24 - DNA, 32 bp, Artificial Sequence
  Description of Artificial Sequence Primer
cgtctagatgacttagtcccagccctcacagg

(19) United States
(12) Patent Application Publication
(10) Pub. No.: US 2006/0127914 A1
(43) Pub. Date: Jun. 15, 2006

(54) Nicotinic acetylcholine receptor

(76) Inventors: Jeffrey Roland Yon, Hoogstraten (BE); Christopher James Grantham, Schoten (BE); Paulus Johannes Groot-Kormelink, London (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/718,933

(22) Filed: Nov. 20, 2003

Related U.S. Application Data
(63) Continuation of application No. 09/661,812, filed on Sep. 14, 2000, now abandoned.
(60) Provisional application No. 60/153,948, filed on Sep. 15, 1999.

(30) Foreign Application Priority Data
Feb. 2, 2000 (GB) GB 0002431.5

(51) Int. Cl. C12Q 1/68; C07H 21/04; C12P 21/06; C07K 14/705; C07D 401/02

(52) U.S. Cl. 435/6; 435/69.1; 435/320.1; 435/325; 530/350; 536/23.5; 546/268.1

(57) ABSTRACT

The invention provides nucleic acid encoding human alpha nicotinic acetylcholine receptor (α10AChR), isolated α10AChR, and assay methods utilising the same.

### FIELD OF THE INVENTION

The present invention relates to a novel human neuronal nicotinic acetylcholine receptor subunit with similarity to the alpha 9 subunit, to nucleic acid encoding it and to its use in assays.

### BACKGROUND OF THE INVENTION

Acetylcholine is a neurotransmitter which activates nicotinic acetylcholine receptors (nAChRs, also referred to herein as AChRs). A number of pathologies and diseases are associated with nAChRs, including myasthenia gravis, schizophrenia, epilepsy, Parkinson's disease, Alzheimer's disease, Tourette's syndrome and nicotine addiction.

Nicotinic acetylcholine receptors are comprised of five subunits, selected from a related family of subunit proteins. The neuronal subunits fall into two main types depending on the presence or absence of a pair of vicinal cysteines close to the binding site for acetylcholine. Thus all α-subunits contain paired cysteine residues thought to play a role in binding of nicotinic agonists (Aplin and Wonnacott, 48, 473-477, 1994) whereas the β subunits do not.

There are nine known alpha subunits, α1 to α9, and at least four beta subunits, β1 to β4. Receptors comprise at least one alpha subunit which in some cell types combine with a beta subunit and in some cases a gamma and delta subunit. For example, the AChR at the neuromuscular junction is believed to have an (α1)₂β1γδ stoichiometry.

Within the group of α subunits there is marked diversity in the manner in which a complete functional nAChR is formed. The majority of the α subunits only form functional receptors when combined as a heteropentamers with β subunits in the CNS (McGehee and Role, Annual Review of Physiology 57, 521-546, 1995). However, α7, α8 and α9 nAChR subunits and the related 5-HT3A subunit are capable of forming functional homopentameric receptors. In this respect it is interesting that the phylogenetic relationship between nAChR subunits suggest that α7, α8, α9 and the related 5-HT3A subunit are more related to each other than to the subunits which only form heteropentameric receptors. Sequence homologies indicate that the α7, α8 and α9 subunits form a distinct subgroup of the alpha subunits.

There is considerable interest in the patent literature in the AChR, and several patent applications describe various family members. For example, WO90/10648 describes the cloning of the rat β4 AChR. WO91/15602 describes clones which encode human α2, α3 and β2 AChR. WO94/20617 describes isolated DNA encoding human α4, α7 and β4 subunits. WO95/13299 also relates to the human α2 subunit, and combinations of it with other named alpha or beta subunits. Human β3 and α6 subunits are described in WO96/41876.

The rat α9 subunit is described by Elgoyhen et al, Cell, 79; 705-715, 1994, and in a related patent application, WO96/03504. The α9 nAChR is unusual in that it has a discrete CNS localisation to sensory neurons (eg. cochlear outer hair cells—Elgoyen et al. (1994); trigeminal ganglia—Liu et al., Brain Research, 809, 238-245, 1998: olfactory bulb—Keiger and Walker, Biochemical Pharmacology, 59, 233-240, 2000). Hybridisation studies have also indicated a possible non-neuronal origin in the pars tuberalis of the pituitary and developing tongue (Elgoyen et al (1994). Also the pharmacology of recombinant α9 diverges from that of other nAChRs. Thus, although α9 is activated by acetylcholine and inhibited by α-bungarotoxin (α-Btx) in common with α7, nicotine behaves as a full antagonist instead of a partial agonist. Moreover, α9 nAChRs are sensitive to some muscarinic, GABAergic, serotonergic and glycinergic agents (Elgoyen et al. 1994; Rothlin et al., Molecular Pharmacology, 55, 248-254, 1999).

The extensive prior art cited above indicates that there is interest in obtaining human AChR subunits. Despite the identification of the rat α9 receptor about six years ago, there were no published reports of a human α9 receptor until a recent clone deposited by Charpantier et al (EMBL/GenBank ID HSA 243342, 1999). This has 91% sequence identity to the rat α9 protein. Prior to this, a number of sequence entries in public domain databases described partial sequences from human cDNA sources said to be similar to the rat α9 sequence, though no full length human clones had been identified.

### SUMMARY OF THE INVENTION

The present invention provides an isolated nucleic acid sequence encoding the polypeptide of SEQ ID NO:2. In a preferred aspect, the isolated sequence is that of SEQ ID NO:1. In another preferred aspect, the isolated nucleic acid sequence is that of SEQ ID NO:3.

The invention also provides a DNA molecule encoding the polypeptide of SEQ ID NO:2, and a DNA molecule encoding the polypeptide of SEQ ID NO:4.

In another aspect, the invention provides an isolated polypeptide having the sequence of SEQ ID NO:2 or SEQ ID NO:4. Polypeptides which are fragments of said polypeptide of SEQ ID NO:2 and SEQ ID NO:4 are also provided, said fragments being of 200 or more amino acids in size. Such fragments may be derived from the N-terminal region of SEQ ID NO:2 or SEQ ID NO:4. Fragments including the N-terminal region may be used to reconstitute the extracellular portion of the receptor to provide receptor binding sites.

In another embodiment, the invention provides an isolated polypeptide having at least 90%, preferably at least 92%, such as at least 95%, more preferably at least 98% or 99% sequence identity to the polypeptide of SEQ ID NO:2 or SEQ ID NO:4. Such polypeptides desirably retain the ability to form a nAchR channel complex that can be activated, amongst others, by acetylcholine. Similarly, polypeptides which are fragments of at least 200 amino acids of these polypeptides having at least 90%, preferably at least 92%, such as at least 95%, more preferably at least 98% or 99% identity to SEQ ID NO:2 or SEQ ID NO:4, including N-terminal fragments, are provided.

In a further aspect, there is provided an isolated polypeptide comprising the mature protein sequence of SEQ ID NO:4, namely residues 25 to the C-terminus of SEQ ID NO:4. Polypeptides having at least 90%, preferably at least 92%, such as at least 95%, more preferably at least 98% or 99% sequence identity to such a polypeptide, preferably those which retain the ability to form nAchR channel complexes, and fragments of at least 200 amino acids thereof are also provided.

Also provided by the invention are isolated nucleic acids encoding the abovementioned polypeptides. Further provided are DNA molecules encoding said polypeptides.

In a further aspect, there are provided vectors comprising the sequences of said nucleic acids, particularly expression vectors comprising a promoter operably linked to the nucleic acid sequences of the invention. The vectors may be carried by a host cell, and expressed within said cell. Following said expression, polypeptides of the invention may be recovered.

In a further aspect, the invention provides assay methods for the identification of substances which bind to or modulate the activity of polypeptides of the invention, either in monomeric or oligomeric (preferably pentameric) form.

The invention also provides antibodies and binding fragments thereof capable of selectively binding to polypeptides of the invention.

These and other aspects of the invention are described herein in more detail.

### BRIEF DESCRIPTION OF THE FIGURES

FIG. 1 illustrates the cloning strategy used to obtain partial α10 clones.

FIG. 2 provides the genomic sequence obtained from cloning experiments. Amino acid sequences in upper case are translated from confirmed exon sequences. The polypeptide coding sequence with homology to the rat α9 receptor is in upper case; this covers the entire predicted mature rat sequence (ie. it extends to the position at which the predicted rat signal peptide would end). It is presumed that there is an upstream splice site which fuses this sequence to a start codon and a signal sequence. Predicted upstream cDNA sequence, intron sequence and 3′UTR are in lower case. Positions of PCR primers used for amplification of unspliced cDNA are shown underlined; * indicates a reverse primer (sequence is inverse complement).

FIG. 3 sets out primers used.

FIG. 4 shows multiple alignment of the human α10 polypeptide (SEQ ID NO:4) with human α9 (Charpantier et al), rat α9 & α10 and chick α10. Sequences were aligned using the CLUSTALW programme (EMBL, Heidelberg, Germany) and visualised with GeneDoc (v2.5.0). Default parameters were used and no further optimization was performed. EMBL database accession numbers of the sequences are as follows: human α9, AJ243342; rat α9, U12336; rat α10, AF196344; chick α10, AJ295624. The rat α9 signal sequence is predicted in the SWISSPROT database to comprise amino acids 1-22, giving a predicted N-terminus of the mature protein of VETAN. The signal prediction algorithm SPScan (Wisconsin Package Version 10.0, Genetics Computer Group (GCG), Madison, Wisc. USA) was used to predict a signal sequence for the human α10 polypeptide: this analysis suggested that the signal comprises amino acids 1-24, giving an N-terminus of the mature protein of AEGRL.

FIG. 5 illustrates the location of various ESTs identified retrospectively.

FIG. 6 shows sequences SEQ ID NO:3 (nucleic acid) and SEQ ID NO:4 (protein).

FIG. 7 shows representative whole cell currents elicited by a 10 s application of ACh (shown as an arrowed index) to Xenopus oocytes injected with either (A—upper panel) α9 alone or (A—lower panel) α9 plus α10. (B) Mean response from 16 to 19 currents from each type of oocyte normalising to the first peak current. (C) Representative examples of the comparison between the current response to a short (10 s) or long (30 s) application of ACh to the α9/α10 combination and to α9 alone.

FIG. 8 shows the concentration-response to ACh of α9 injected alone into oocytes (open symbols) compared with injection of the α9/α10 combination (filled symbols). Various concentrations of ACh were applied for 10 s bracketed between control applications of 30 μM ACh. 5 minutes washout time was allowed between each response. The data is corrected for run-up or run-down and expressed as a percentage of the maximum evoked current. Curves have been fitted using the non-linear curve fitting routine of Graph Pad. The fit is constrained to a lower bound of 0 while the upper bound is unconstrained. The correlation coefficients are R²=0.9999 and 0.9980 for α9 and α9/α10, respectively.

FIG. 9 illustrates inhibition of ACh-evoked responses in α9 injected alone into oocytes (open symbols) compared with injection of the α9/α10 combination (filled symbols) by α-Btx. The oocytes were incubated with various concentration of α-Btx for 2 minutes prior to a 10 s application of ACh. Nicotinic responses were evoked with ACh at concentrations corresponding to the approximate EC50s for either type of oocyte (e.g 25 μM for α9 or 50 mM for (α9/α10). Each application of α-Btx was bracketed between control applications of ACh in the absence of toxin. 5 minutes washout time was allowed between each response. The data is expressed as a percentage of the control current and curves have been fitted using the non-linear curve fitting routine of Graph Pad. The fit is constrained to upper and lower bounds of 100 and 0. The correlation coefficients are R²=0.9973 and 0.9931 for α9 and α9/α10, respectively.

### DETAILED DESCRIPTION OF THE INVENTION

The present inventors have now cloned a novel human AChR α subunit, which is distinct from the α9 subunit, though related to it—with 56% sequence identity. Although initial data—generated prior to the database entry of Charpantier et al and disclosed in U.S. Patent Application 60/153,948 filed 15 Sep. 1999—suggested that this may have been α9, we have now termed the novel subunit α10, to distinguish it from the newly found α9. From the unusual distribution of this subunit together with its apparent ability to co-assemble with the α9 nAChR subunit we propose that α10 contributes to cholinergic transmission both in the CNS and in certain non-neuronal tissues with importance to the hormonal and immunological status of the organism.

The production of the α10 subunit is all the more surprising in that it was unexpectedly identified in an attempt to generate a human α9-like subunit. In this attempt, a number of unexpected obstacles were encountered, including failure of regions of the sequence to extend in PCR and failure of primary positives cloned in E. coli systems to propagate. Although the reasons for this are not apparent, the failure of the art to date to provide a human α9 clone suggests that the gene may contain sequences which had at least until late in 1999 thwarted others in the art from succeeding. A recent entry on the EMBL database, AF196344, submitted on 19 Oct. 1999, provides a sequence indicated to be a rat α10 AChR encoding mRNA. The predicted ORF has 91% amino acid identity to SEQ ID NO:4. A further entry on the EMBL database, AJ295624, submitted on 24 Jul. 2000 provides a sequence indicated to be a chick α10 AChR encoding mRNA.

Nucleic Acid

Nucleic acid includes DNA (including both genomic and cDNA) and RNA. Where nucleic acid according to the invention includes RNA, reference to the sequences shown in the accompanying listings should be construed as reference to the RNA equivalent, with U substituted for T.

Nucleic acid of the invention may be single or double stranded. Single stranded nucleic acids of the invention include anti-sense nucleic acids. Thus it will be understood that reference to SEQ ID NO:1 or SEQ ID NO:3 or sequences comprising SEQ ID NO:1 or SEQ ID NO:3 or fragments thereof include complementary sequences unless the context is clearly to the contrary.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA.

The invention also provides nucleic acids which are fragments of the nucleic acids encoding a polypeptide of the invention. In one aspect, the invention provides nucleic acids primers which consist essentially of from 15 to 50, for example from 15 to 35, 18 to 35, 15 to 24, 18 to 30, 18 to 21 or 21 to 24 nucleotides of a sequence encoding a polypeptide of the invention or its complement.

The term “consist essentially of” refers to nucleic acids which do not include any additional 5′ or 3′ nucleic acid sequences. In a further aspect of the invention, nucleic acids of the invention which consist essentially of from 15 to 30 nucleotides as defined above may however be linked at the 3′ but preferably 5′ end to short (e.g from 4 to 15, such as from 4 to 10 nucleotides) additional sequences to which they are not naturally linked. Such additional sequences are preferably linkers which comprise a restriction enzyme recognition site to facilitate cloning when the nucleic acid of the invention is used for example as a PCR primer.

Primers of the invention are desirably capable of selectively hybridising to nucleic acids encoding the polypeptides of the invention. By “selective”, it is meant selective with respect to other alpha subunit sequences in humans and the alpha 9 and other alpha subunit sequences in rats. Primers which are derived from SEQ ID NO:3 not present in SEQ ID NO:1 will also be capable of selectively hybridising to SEQ ID NO:3 compared to the human α9 sequence of Charpantier et al. The ability of the sequence to hybridise selectively may be determined by experiment or calculated.

For example, one way to calculate Tm of a primer is by reference to the formula for calculating the Tm of primers to a homologous target sequence. This formula is Tm(° C.)=2(A+T)+4(G+C)−5. This will provide the Tm under conditions of 3×SSC and 0.1% SDS (where SSC is 0.15M NaCl, 0.015M sodium citrate. pH 7). This formula is generally suitable for primers of up to about 50 nucleotides in length. In the present invention, this formula may be used as an algorithm to calculate a nominal Tm of a primer for a specified sequence derived from a sequence encoding a polypeptide of the invention. The Tm may be compared to a calculated Tm for other alpha subunit sequences of humans and rats, based upon the maximum number of matches to any part of these other sequences.

Thus in a preferred aspect, a primer of the present invention will have a Tm (calculated as above) for a sequence encoding a polypeptide of the invention which is at least 5° C. higher than for the other alpha subunit encoding sequences. Preferably the difference is at least 8° C., more preferably at least 10° C., at least 15° C. or at least 20° C. (Since for the purposes of the present invention the above formula is used as an algorithm, the actual Tm of primers when hybridised to non-complementary targets which do not exactly match the primer sequence may or may not correspond to the calculated value.)

Suitable conditions for a primer to hybridise to a target sequence may also be measured experimentally. Suitable experimental conditions comprise hybridising a candidate primer to both nucleic acid encoding a polypeptide of the invention and nucleic acid encoding other alpha subunits on a solid support under low stringency hybridising conditions (e.g. 6×SSC at 55EC), washing at reduced SSC and/or higher temperature, for example at 0.2×SSC at 45EC, and increasing the hybridisation temperature incrementally to determine hybridisation conditions which allow the primer to hybridise to nucleic acid encoding a polypeptide of the invention but not other alpha subunit encoding nucleic acids.

Nucleic acids of the invention, particularly primers may carry a revealing label. Suitable labels include radioisotopes such as ³²P or ³⁵S, fluorescent labels, enzyme labels, or other protein labels such as biotin. Such labels may be added to polynucleotides or primers of the invention and may be detected using by techniques known per se.

Primers of the present invention may be comprises of synthetic nucleic acids, such as those with modified backbone structures intended to improve stability of the nucleic acid in a cell. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention.

Also included within the scope of the invention are antisense sequences based on the nucleic acid sequences described herein, preferably in the form of oligonucleotides, particularly stabilized oligonucleotides, or ribozymes.

Antisense oligonucleotides may be designed to hybridise to the complementary sequence of nucleic acid, pre-mRNA or mature mRNA, interfering with the production of polypeptide encoded by a given target DNA sequence, so that its expression is reduced or prevented altogether. Ribozymes will be designed to cleave mRNA encoded by an α10AChR encoding nucleic acid sequence of the invention, desirably at a target sequence specific to the α10AChR sequence, i.e one which is not common to other AChR sequences. The construction of antisense sequences and their use is described in Peyman and Ulman, Chemical Reviews, 90:543-584, (1990); Crooke, Ann. Rev. Pharmacol. Toxicol., 32:329-376, (1992), and Zamecnik and Stephenson, P.N.A.S, 75:280-284, (1974). The construction of ribozymes and their use in described in for instance Gibson and Shillitoe, Molecular Biotechnology 7(2): 125-137, (1997).

Antisense and ribozyme sequences of the invention may be introduced into mammalian cells lines in culture to study the function of α10AChR, for example by causing down-regulation of this gene and observing phenotypic effects, or the expression or location of proteins described herein which associate with α10AChR. In cells where aberrant expression of α10AChR occurs, such antisense and ribozyme sequences may be used to down-regulate the expression of the gene.

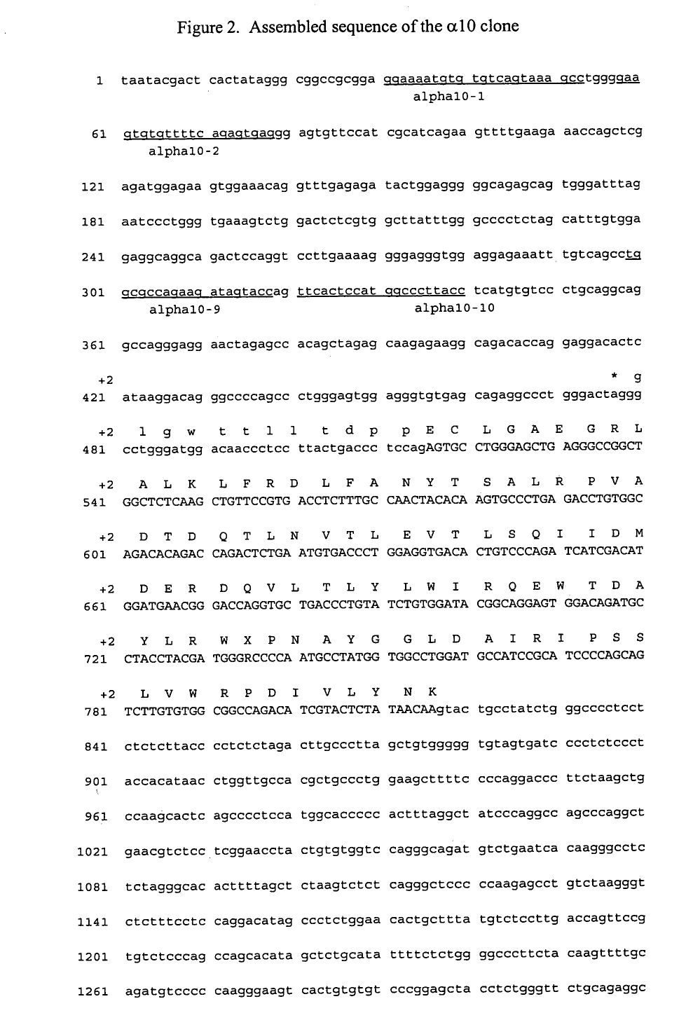

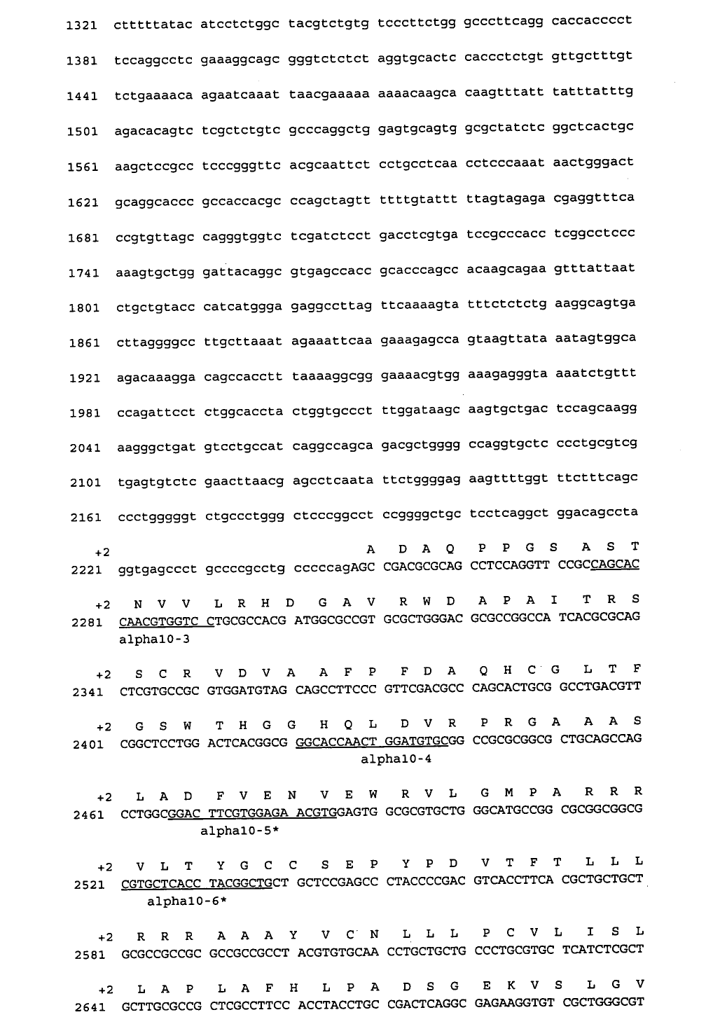

Nucleic acid sequences encoding SEQ ID NO:1 or SEQ ID NO:3 may be prepared by reference to the accompanying examples. The examples illustrate that the partially spliced message encoding the α10AChR subunit may be relatively abundant compared to the fully spliced message, and that in order to obtain a sequence encoding the polypeptide of SEQ ID NO:2 or SEQ ID NO:4 it may be necessary to assemble the sequence from partial overlapping fragments. This may be achieved by PCR amplification of separate regions of the sequence, e.g. the separate exon sequences shown in FIG. 2, which may then be spliced together using conventional techniques.

FIG. 2 indicates that the 5′ terminal sequence encoding the first of the two exons illustrated is missing the three N-terminal amino acids of SEQ ID NO:2. The sequence encoding them may be introduced artificially by PCR techniques into an amplified produce comprising this exon.

Polynucleotides which are not 100% homologous to the sequence of SEQ ID NO:1 or SEQ ID NO:3 but which encode either SEQ ID NO:2 or SEQ ID NO:4 or other polypeptides of the invention can be obtained in a number of ways.

For example, site directed mutagenesis of the sequences of SEQ ID NO. 1 or SEQ ID NO:3 may be performed. This is useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Further changes may be desirable to represent particular coding changes which are required to provide, for example, conservative substitutions.

Nucleic acids of the invention may comprise additional sequences at the 5′ or 3′ end. For example, synthetic or natural 5′ leader sequences may be attached to the nucleic acid encoding polypeptides of the invention. The additional sequences may also include 5′ or 3′ untranslated regions required for the transcription of nucleic acid of the invention in particular host cells.

The present invention further extends to an isolated DNA sequence comprising sequences encoding a polypeptide of the invention but in which the encoding sequences are divided up into two or more (preferably no more than five, e.g. four or three) exons. An example of such a DNA sequence is shown in FIG. 2. Such exon sequences may be natural and obtained from genomic clones, or synthetic. Exon sequences may be used in the construction of mini-gene sequences which comprise nucleic acid encoding polypeptides of the invention which sequences are interrupted by one or more exon sequences.

In one aspect of the invention, there is provided nucleic acid comprised of the second and third exons of FIG. 2, the exons being either joined or separated by an intron, and an isolated polypeptide comprised of the sequence encoded by such a nucleic acid.

Mini-genes may also be constructed using heterologous exons, derived from any eukaryotic source.

Nucleic acid according to the present invention, such as a full-length coding sequence or oligonucleotide probe or primer, may be provided as part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc.

The nucleic acid of the invention may be used in nucleic acid-based tests for detecting the α10AChR encoding sequences in the human body or tissues or samples obtained therefrom. In the case of detecting, this may be qualitative and/or quantitative, including such methods as microarray technology on a DNA chip. Detection includes analytical steps such as those which involve sequencing the gene in full or in part.

Such tests for detecting generally comprise bringing a human sample containing DNA or RNA into contact with a probe comprising a nucleic acid of the invention or primer of the invention under hybridizing conditions and detecting any duplex formed between the probe and nucleic acid in the sample. Such detection may be achieved using techniques such as PCR or by immobilizing the probe on a solid support, removing nucleic acid in the sample which is not hybridized to the probe, and then detecting nucleic acid which has hybridized to the probe. Alternatively, the sample nucleic acid may be immobilized on a solid support, and the amount of probe bound to such a support can be detected. Suitable assay methods of this and other formats can be found in for example WO89/03891 and WO90/13667.

A further method of detection according to the invention is in detecting changes to wild-type α10AChR genes, including single base changes, for example using single stranded conformational polymorphism (SSCP) analysis. Nucleic acid sequence from all or part of an α10AChR encoding DNA or mRNA in a sample may be hybridized to a reference sequence, and the mobility of the hybrid is observed in a gel under conditions where any non-hybridized regions within the duplex give rise to changes in mobility.

The nucleic acids of the present invention are also useful in tissue distribution studies, to confirm and extend the knowledge of this gene's distribution. Our experiments have shown that the gene is expressed in a variety of tissue types, including pituitary gland, lymphomas, liver, lung, peripheral blood leukocytes, tongue, testis and spleen.

Rat α9AChR has also been found in the cochlea, trigeminal ganglia and olfactory lobe, samples of these tissues type may also be examined, given the similarities between the α9 and α10 sequences.

Polypeptides

Isolated polypeptides of the invention will be those as defined above in isolated form, free or substantially free of material with which it is naturally associated such as other polypeptides with which it is found in the cell. The polypeptides may of course be formulated with diluents or adjuvants and still for practical purposes be isolated—for example the polypeptides will normally be mixed with gelatin or other carriers if used to coat microtitre plates for use in immunoassays. The polypeptides may be glycosylated, either naturally or by systems of heterologous eukaryotic cells, or they may be (for example if produced by expression in a prokaryotic cell) unglycosylated. Polypeptides may phosphorylated and/or acetylated.

A polypeptide of the invention may also be in a substantially purified form, in which case it will generally comprise the polypeptide in a preparation in which more than 90%, e.g. 95%, 98% or 99% of the polypeptide in the preparation is a polypeptide of the invention.

Polypeptides of the invention may be modified for example by the addition of histidine residues to assist their purification or by the addition of a signal sequence to promote their secretion from a cell.

Polypeptides having at least 90% sequence identity, for example at least 95%, 98% or 99% sequence identity to SEQ ID NO:2 or SEQ ID NO:4 may be polypeptides which are amino acid sequence variants, alleles, derivatives or mutants of SEQ ID NO:2 or SEQ ID NO:4, and are also provided by the present invention. For example such a polypeptide may have an amino acid sequence which differs from that given in SEQ ID NO:2 or SEQ ID NO:4 by one or more of addition, substitution, deletion and insertion of one or more (such as from 1 to 20, for example 2, 3, 4, or 5 to 10) amino acids.

The percentage identity of polypeptide sequences can be calculated using commercially available algorithms which compare a reference sequence (in the present invention SEQ ID NO:2 or SEQ ID NO:4) with a query sequence. The following programs (provided by the National Center for Biotechnology Information) may be used to determine homologies: BLAST, gapped BLAST, BLASTN and PSI-BLAST, which may be used with default parameters.

The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4. Use of either of the terms “homology” and “homologous” herein does not imply any necessary evolutionary relationship between compared sequences, in keeping for example with standard use of terms such as “homologous recombination” which merely requires that two nucleotide sequences are sufficiently similar to recombine under the appropriate conditions.

Another method for determining the best overall match between a nucleic acid sequence or a portion thereof, and a query sequence is the use of the FASTDB computer program based on the algorithm of Brutlag et al (Comp. App. Biosci., 6; 237-245 (1990)). The program provides a global sequence alignment. The result of said global sequence alignment is in percent identity. Suitable parameters used in a FASTDB search of a DNA sequence to calculate percent identity are: Matrix=Unitary, k-tuple=4, Mismatch penalty=1, Joining Penalty=30, Randomization Group Length=0, Cutoff Score=1, Gap Penalty=5, Gap Size Penalty=0.05, and Window Size=500 or query sequence length in nucleotide bases, whichever is shorter. Suitable parameters to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in nucleotide bases, whichever is shorter.

Where a query sequence is determined to have an identity to that of SEQ ID NO:2 or SEQ ID NO:4 of at least 90%, preferably at least 92%, such as at least 95%, more preferably at least 98% or 99%, said sequence being that of a polypeptide retaining activity as a ligand-gated ion channel capable of activation by acetylcholine or other nicotinic agonists, such a sequence forms part of the present invention. Such properties of nAChRs are described in Colquhoun, L. M. and Patrick, J. W., Advances in Pharmacology (New York) 39: 191-220, 1997, which is incorporated herein by reference.

Polypeptides of the invention include fragments of SEQ ID NO:2 or SEQ ID NO:4 which are encoded by the exons of the gene encoding the alpha 10 receptor. Such fragments include a polypeptide comprising the third exon-encoded polypeptide shown in FIG. 2, the second exon-encoded polypeptide shown in FIG. 2, and a polypeptide comprising the second and third exon-encoded polypeptides linked directly to each other. Variants of such polypeptides having at least 90%, e.g at least 92, such as at least 95%, more preferably at least 98% or 99% identity to said exon-encoded polypeptides also form part of the present invention.

A variant of the second exon-encoded polypeptide contemplated by the invention is one which has an alternative N-terminal extension of the sequence. This polypeptide may also be attached to the third exon-encoded polypeptide.

A polypeptide according to the present invention may be isolated and/or purified (e.g. using an antibody) for instance after production by expression from encoding nucleic acid. The isolated and/or purified polypeptide may be used in formulation of a composition, which may include at least one additional component, for example a pharmaceutical composition including a pharmaceutically acceptable excipient, vehicle or carrier.

A polypeptide according to the present invention may be used as an immunogen or otherwise in obtaining specific antibodies. Antibodies are useful in purification and other manipulation of polypeptides, diagnostic screening and therapeutic contexts. This is discussed further below.

A polypeptide according to the present invention may be used in screening for molecules which bind to it or modulate its activity or function. Such molecules may be useful in a therapeutic (possibly including prophylactic) context.

A polypeptide of the invention may be labelled with a revealing label. The revealing label may be any suitable label which allows the polypeptide to be detected. Suitable labels include radioisotopes, e.g. ¹²⁵I, enzymes, antibodies, fluorescent dyes, polynucleotides and linkers such as biotin. Labelled polypeptides of the invention may be used in diagnostic procedures such as immunoassays in order to determine the amount of a polypeptide of the invention in a sample. Polypeptides or labelled polypeptides of the invention may also be used in serological or cell mediated immune assays for the detection of immune reactivity to said polypeptides in animals and humans using standard protocols.

A polypeptide or labelled polypeptide of the invention or fragment thereof may also be fixed to a solid phase, for example the surface of an immunoassay well or dipstick.

Such labelled and/or immobilized polypeptides may be packaged into kits in a suitable container along with suitable reagents, controls, instructions and the like.

Such polypeptides and kits may be used in methods of detection of antibodies to such polypeptides present in a sample or active portions or fragments thereof by immunoassay.

Immunoassay methods are well known in the art and will generally comprise:
(a) providing a polypeptide comprising an epitope bindable by an antibody against said protein;
(b) incubating a biological sample with said polypeptide under conditions which allow for the formation of an antibody-antigen complex; and
(c) determining whether antibody-antigen complex comprising said polypeptide is formed.
   Antibodies

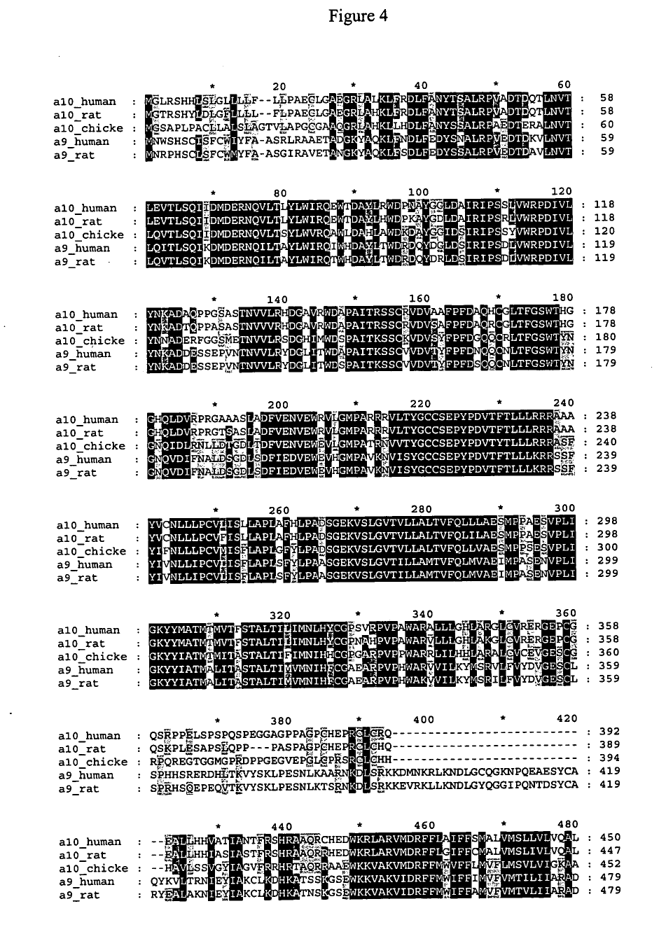

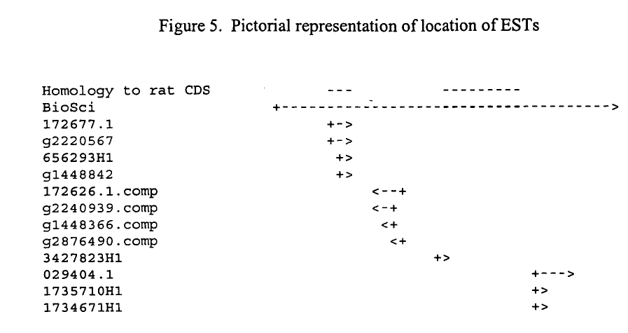

The provision of the polypeptides of the invention enables for the production of antibodies able to bind human α10AChR in a specific manner. Thus the invention provides an antibody which is able to bind specifically to a polypeptide of the invention and not to the rat α9AChR. Such an antibody will have an affinity for a polypeptide of the invention of at least 100 fold, preferably at least 1000 fold more than to the rat α9AChR. Such antibodies may be produced using epitopes of polypeptides of the invention which are not present in the rat AChR. Such epitopes can be determined by seeking differences between polypeptides of the invention and the rat AChR sequence, for example the sequence shown in FIG. 4. In a further preferred aspect, the antibodies will be capable of distinguishing between the alpha subunit sequence of Charpantier et al cited above and the protein of SEQ ID NO:2 or SEQ ID NO:4 by binding specifically to polypeptides of the invention, the differential binding affinity being as defined above.

Antibodies may be obtained using techniques which are standard in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

The mode of determining binding is not a feature of the present invention and those skilled in the art are able to choose a suitable mode according to their preference and general knowledge.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid therefor. Antibodies may modulate the activity of the polypeptide to which they bind and so, if that polypeptide has a deleterious effect in an individual, may be useful in a therapeutic context (which may include prophylaxis).

An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

Vectors

Nucleic acid sequences of the present invention may be incorporated into vectors, particularly expression vectors. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

Preferably, a polynucleotide of the invention in a vector is operably linked to a control sequence which is capable of providing for the expression of the coding sequence by the host cell, i.e. the vector is an expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage phagemid or baculoviral, cosmids, YACs, BACs, or PACs as appropriate. Vectors include gene therapy vectors, for example vectors based on adenovirus, adeno-associated virus, retrovirus (such as HIV or MLV) or alpha virus vectors.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which is can be included in response to heavy metals such as cadmium. Viral promoters such as the SV40 large T antigen promoter or adenovirus promoters may also be used. All these promoters are readily available in the art.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell.

Vectors for production of polypeptides of the invention of for use in gene therapy include vectors which carry a mini-gene sequence of the invention.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1997.

Vectors may be transformed into a suitable host cell as described above to provide for expression of a polypeptide of the invention. Thus, in a further aspect the invention provides a process for preparing polypeptides according to the invention which comprises cultivating a host cell transformed or transfected with an expression vector as described above under conditions to provide for expression by the vector of a coding sequence encoding the polypeptides, and recovering the expressed polypeptides. Polypeptides may also be expressed in in vitro systems, such as reticulocyte lysate.

A further embodiment of the invention provides host cells transformed or transfected with the vectors for the replication and expression of polynucleotides of the invention. The cells will be chosen to be compatible with the said vector and may for example be bacterial, yeast, insect or mammalian. The host cells may be cultured under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers

Polynucleotides according to the invention may also be inserted into the vectors described above in an antisense orientation in order to provide for the production of antisense RNA or ribozymes.

Assays

The present invention also provides a method for identifying compounds which bind to the human α10AChR subunit polypeptides of the present invention, either alone or in the form of a receptor including other subunits of the nAchR alpha or beta class or including 5HT₃ subunits. In such a method, the receptor subunits may be employed in a competitive binding assay. Such an assay can accommodate the rapid screening of a large number of compounds to determine which compounds, if any, are capable of binding to the subunit polypeptides. Subsequently, more detailed assays can be carried out with those compounds found to bind, to further determine whether such compounds act as agonists or antagonists of the polypeptides of the invention, the polypeptides being either isolated monomeric polypeptides or homopolymeric receptors, or in the form of a receptors including other subunits of the nAChR alpha or beta class or including 5HT₃ subunits.

The present invention still further provides a bioassay for identifying compounds which modulate the activity of receptors comprising polypeptides of the invention. In one embodiment, the bioassay is conducted by providing cells expressing monomeric polypeptides or polymeric receptors comprising at least one subunit comprising a polypeptide of the invention with at least one potential agonist and thereafter monitoring the cells for changes in ion channel activity. In yet another embodiment, the bioassay is conducted by contacting cells expressing at least one receptor subunit comprising a polypeptide of the invention with a constant amount of known α10 agonist, including ACh and increasing amounts of at least one potential antagonist and thereafter monitoring the cells for changes in ion channel activity.

Suitable cells include insect, amphibian or mammalian cells. Xenopus oocytes are suitable for this purpose.

The present invention also provides a bioassay for identifying compounds which modulate the regulatory regions of the human α10AChR gene. Such an assay is conducted utilising human cells capable of expressing the polypeptide of the invention (preferably of SEQ ID NO:2 or SEQ ID NO:4). The cells are contacted with at least one compound wherein the ability of said compound to modulate the regulatory region is known.

Thereafter, the cells are monitored for expression of the nucleic acid of the invention. Alternatively, the promoter may be linked to a reporter gene. Suitable reporter genes that may be employed include, for example, the chloramphenicol acetyltransferase gene, the luciferase gene, and the like.

A compound or a signal that “modulates the activity” of a polypeptide of the invention refers to a compound or a signal that alters the activity of the polypeptide so that it behaves differently in the presence of the compound or signal than in the absence of the compound or signal. Compounds affecting modulation include agonists and antagonists. An agonist encompasses a compound such as acetylcholine, that activates α10 containing receptor function. Alternatively, an antagonist includes a compound that interferes with α10 containing receptor function. Typically, the effect of an antagonist is observed as a blocking of agonist-induced receptor activation. Antagonists include competitive as well as non-competitive antagonists. A competitive antagonist (or competitive blocker) interacts with or near the site specific for agonist binding. A non-competitive antagonist or blocker inactivates the function of the receptor by interacting with a site other than the agonist interaction site.

As understood by these of skill in the art, bioassay methods for identifying compounds that modulate the activity of receptors such as polypeptides of the invention generally require comparison to a control. One type of “control” is a cell or culture that is treated substantially the same as the test cell or test culture exposed to the compound, with the distinction that the “control” cell or culture is not exposed to the compound. For example, in methods that use voltage clamp electrophysiological procedures, the same cell can be tested in the presence or absence of compound, by merely changing the external solution bathing the cell. Another type of “control” cell or culture that can be employed is a cell or culture that is identical to transfected cells, the exception that the “control” cell or culture does not express functional α10 containing AChR subunit. Accordingly, the response of the transfected cell to the “control” cell or culture to the same compound under the same reaction conditions.

In another aspect, the ion channel activity of the polypeptides of the invention may be modulated by contacting said polypeptides with at least one compound identified by any of the assay methods of the present invention.

Where assays of the invention involve testing compounds with polypeptides of the invention, the assays may also involve testing the compound with α9 polypeptides. In such assays, the α9 polypeptides may be expressed in the same cells as the polypeptides of the invention, may be provided in different cells within the same preparation as the cells expressing the polypeptides of the invention, or may be provided in separate parallel preparations. In such assays, the ability of the compound to modulate the activity of the polypeptides of the invention in the presence of the alpha9 polypeptides may be determined and the ability compared with the ability of the compound to modulate the activity of the alpha9 polypeptides in the absence of the polypeptides of the invention.

Particularly preferred types of assays include binding assays and functional assays which may be performed as follows:

Binding Assays

Over-expression of nucleic acid encoding polypeptides of the invention in cell lines (including mammalian HEK 293 cells and Sf9 insect cells) may be used to produce membrane preparations bearing said polypeptides (referred to in this section as α10 nAChR for convenience) for ligand binding studies. These membrane preparations can be used in conventional filter-binding assays (eg. Using Brandel filter assay equipment) or in high throughput Scintillation Proximity type binding assays (SPA and Cytostar-T flashplate technology; Amersham Pharmacia Biotech) to detect binding of radio-labelled nicotinic ligands (including ³H- or ¹⁴C-acetylcholine, epibatidine, methyllycaconitine, ¹²⁵ I-α-bungarotoxin) and displacement of such radio-ligands by competitors for the binding site. Radioactivity can be measured with Packard Topcount, or similar instrumentation, capable of making rapid measurements from 96-, 384-, 1536-microtitre well formats. SPA/Cytostar-T technology is particularly amenable to high throughput screening and therefore this technology is suitable to use as a screen for compounds able to displace standard ligands.

Alternative methods are also available for measuring ligand-binding, making use of fluorescence. Newly developed fluorescence ligands of high emission intensity (eg. fluorescently-labelled α-bungarotoxin) may be used to detect α10 nAChR protein in membrane preparations and displacement of such fluorescent ligands by competitors for the binding site. Fluorescence can be measured with LJL Analyst or similar technology in 96-, 384- or 1536-well microtitre formats.

Another approach is to image fluorescence-based ligand binding in whole fixed or living cells giving the advantage of being able to study α10 nAChR protein in an environment and conformation, either approximating, or mimicking that of the native receptor. These techniques can be used to quantify α10 nAChR protein occurrence in recombinant cell lines and to examine competition for the binding site, by agents of interest in kinetic or end-point assays using fluorescence polarisation. Fluorescence polarisation measurements can be made using LJL Analyst and Acquest and/or BMG Polarstar fluorescence plate readers or other similar technology.

Another approach to study binding of ligands to α10 nAChR protein in an environment approximating the native situation makes use of a surface plasmon resonance effect exploited by the Biacore instrument (Biacore). α10 nAChR in membrane preparations or whole cells could be attached to the biosensor chip of a Biacore and binding of ligands including α-bungarotoxin examined in the presence and absence of compounds to identify competitors of the binding site.

Functional Assays

Since nAChRs are ligand-gated cation channels, they will allow cations to pass through when they are activated by acetylcholine and other nicotinic agonists. Sodium and calcium will flux in and potassium ions will flux out of the cells, according to Nernstian principles. This flux of ions is an essential component of the function (eg. signal transduction system) of nAChRs. Influx of sodium and calcium depolarises the membrane potential and thereby will influence voltage-sensitive processes within the cell. Calcium entry into cells is an important signal for enabling neurotransmitter release and mediating nuclear processes at the gene transcription level. It is possible to measure this flux of ions in real time using a variety of techniques.

Electrophysiology

Flux of positive ions through nAChRs give rise to a current, which can be measured using electrophysiological methods. Therefore, recombinant α10 containing nAChRs expressed in cell lines can be characterised using whole cell and signal channel electrophysiology to determine the mechanism of action of compounds of interest. Electrophysiological screening, for compounds active at α10 containing nAChRs, may be performed using conventional electrophysiological techniques and when they become available, novel high throughput methods currently under development.

Fluorescence

Calcium and sodium fluxes are measurable using several ion-sensitive fluorescent dyes, including fluo-3, fluo-4, fluo-5N, fura red, Sodium Green, SBFI and other similar probes from suppliers including Molecular Probes. Other fluorescent dyes, from suppliers including Molecular Probes, such as DIBAC_{4 (3)} or Di-4-Anepps can detect membrane potential changes. Calcium and sodium influx through α10 containing nAChRs can thus be characterised in real time, using fluorometric and fluorescence imaging techniques, including fluorescence microscopy with or without laser confocal methods combined with image analysis algorithms.

Another approach is a high throughput screening assay for compounds active as either agonists or modulators of ligand-gated ion channels which flux calcium. This assay is based around an instrument called a FLuorescence Imaging Plate Reader ((FLIPR), Molecular Devices Corporation). In its most common configuration, it excites and measures fluorescence emitted by fluorescein-based dyes. It uses an argon-ion laser to produce high power excitation at 488 nm of a fluorophore, a system of optics to rapidly scan the over the bottom of a 96-/384-well plate and a sensitive, cooled CCD camera to capture the emitted fluorescence. It also contains a 96-/384-well pipetting head allowing the instrument to deliver solutions of test agents into the wells of a 96-/384-well plate. The FLIPR assay is designed to measure fluorescence signals from populations of cells before, during and after addition of compounds, in real time, from all 96-/384-wells simultaneously. The FLIPR assay may be used to screen for and characterise compounds functionally active at recombinant human α10 containing nAChRs expressed in cell lines. With modification it may be possible to use this system to measure sodium fluxes through recombinant human α10 containing nAChRs expressed in cell lines.

Radioactive Methods

⁸⁶-Rubidium and ¹⁴C-guanidinium are useful radiolabels with which to measure non-specific cation channel function in cell-based systems. If the cells are pre-loaded with ⁸⁶-rubidium (which substitutes for potassium), or are bathed in buffer, containing ¹⁴C-guanidinium (as a substitute for sodium), they will flux through any channel (e.g nAChRs) allowing passage of potassium or sodium. The net result is that activation of the receptor will result in either loss of ⁸⁶-rubidium or accumulation of ¹⁴C-guanidinium by the cells. This change in cellular radioactivity is measurable by SPA and Cytostar-T flashplate technology. Therefore, such assay systems may be used as a basis to screen for compounds active at recombinant human α10 containing nAChRs expressed in cell lines.

Cell Adhesion/Migration Assay

α10 nAChR subunit sequence appears to be associated with leukocytes. While not wishing to be bound by any one particular theory, it is believed that there may be a significant association between α10 containing nAChRs, leukocytes and activation processes leading to immunological or inflammatory events. Thus another assay format is to measure the activity of recombinant human α10 containing nAChRs over-expressed in leukocytic tumour cell lines, including HL60 cells. The assay will characterise the α10 containing nAChR activation with respect to cell adhesion/migration properties in the recombinant leukocytic tumour cell lines. Leukocytic tumour cells are grown within microporous filter inserts (eg. Costar Transwells, or Falcon HTS Fluoroblok inserts which have pores of a size to allow activated leukocytic cells to pass through, but not unactivated leukocytic cells) carried in 24-/96-well plates. Residual adhesion/migration of leukocytic cells over-expressing α10 containing nAChRs will be assessed in comparison with sham-transfected leukocytic cells. The effect of acetylcholine and other nicotinic agonists and antagonists is then determined in this system. Established stimuli of leukocytic adhesion/migration (including mediators such as the interleukins, endotoxins, leukotrines, prostaglandins, TNFs) may be applied in this assay to look for modulatory effects of standard nicotinic agonists or antagonists. The assay may further be used to screen for compounds active at down-regulating adhesion/migration of leukocytic tumour cells. Thus if acetylcholine enhances adhesion/migration of leukocytic tumour cells, the assay can be designed to find selective α10 antagonists. If acetylcholine itself down-regulates adhesion/migration in leukocytic tumour cells, then the assay can be designed to find agonists selective for α10 containing nAChRs.

Adhesion/migration will be assessed by recovery of leukocytic tumour cells from inside the microporous filters, after washing (adhesion) or from the buffer outside of the microporous insert (migration). Quantitation may be by total protein, or DNA determination (eg. Hoechst stain), or eosinophil peroxidase, or myeloperoxidase, major basic protein determination using specific coloured substrate reagents, or other assay formats such as ELISA.

Compounds found to modulate the activity of the polypeptides of the present invention have a number of therapeutic uses. For example, the occurrence of α10AchR subunits in leukocytes may indicate a role in immune function and/or inflammation. In particular, the association with lung could suggest a role in asthma as leukocytes such as activated eosinophils and neutrophils accumulate in the lungs and transmigrate into the alveoli in this disease. Acetylcholine is a major neurotransmitter which activates nAChRs. The lung possesses an extensive sensory nervous supply and accumulations of activated leukocytes are often associated with sensory neurons in inflammatory lung diseases like asthma. It is well known that acetylcholine release is increased during episodes of asthma and therefore acetylcholine release from such nerve terminals could be involved in directly modulating the inflammatory process through α10 containing AChRs on leukocytes. Although exogenous administration of acetylcholine can provoke an asthma attack the direct effect of acetylcholine on leukocyte infiltration and activation is not yet known. If acetylcholine stimulates leukocytic activation then a selective α10 containing AChR antagonist would be a potential novel anti-asthma agent, helping to prevent lung infiltration of eosinophils and neutrophils. If, on the other hand, acetylcholine inhibits leukocytic activation then a selective α10 containing AChR agonist may be a potential novel anti-asthma agent for the same reason, without having the side effects of acetylcholine such as precipitation of an asthma attack.

The potential use of selective α10 containing AChR modulatory agents may be extended to the therapy of important inflammatory diseases including chronic obstructive lung disease, acute adult respiratory distress syndrome, sepsis, rheumatoid and osteo-arthritis, inflammatory bowel syndrome, Cröhn's disease and psoriasis.

Further, the finding that α10AchR subunit is also distributed in CNS tissues suggests that selective modulators of α10 containing AChR could have potential therapeutic value in CNS diseases. There are precedents in other nAChRs such as α7 containing AChR for therapy of neurodegenerative diseases such as Alzheimer's and Parkinson's disease, and pyschotic diseases such as schizophrenia, by selective agonists of this receptor. nAChRs in general have been implicated in other CNS pathologies such as epilepsy and centrally mediated chronic pain.

Localisation in pituitary tissue may indicate a major role in endocrine regulation. It has been suggested that α7 containing nAChRs are ligand-gated calcium channels, capable of influencing excitatory neurotransmitter release in the CNS. By analogy, the α10 containing nAChR may have a role in direct release into the blood stream of neurohypophysial hormones such as CRF, vasopressin and oxytocin. Alternatively, α10 nAChR subunits localised to the adenohypophysis could be involved in release of potent hormones such as ACTH, prolactin, and growth hormone. The actions of such hormones are diverse, ranging from preparing and coping with stress, milk formation and ejection in suckling females, control of blood pressure and more subtle effects on memory, anxiety and depression. Selective agonists of α10 containing nAChRs may, therefore, have therapeutic value in countering diseases caused by hormonal deficiencies. Alternatively, diseases caused by over-production of hormones may be controlled with selective antagonists of α10 containing nAChRs.

Tissue Distribution Studies

Human α10 DNA sequences are useful for confirming and extending knowledge of this nAChR=s distribution by hybridisation/PCR studies in human health and disease. Recombinant α10 containing nAChRs in cell lines are useful for characterising α10 nAChR function and a variety of biochemical and biophysical methods are available for assaying this receptor. Assays of the invention described herein are also useful for drug discovery screening purposes.

Binding Agents

Thus the invention further provides novel binding agents, including modulatory agents obtained by an assay according to the present invention, and compositions comprising such agents. Agents which bind to the receptor and which may have agonist or antagonist activity may be used in methods of treating diseases whose pathology is characterised by action via the α10ACh receptor, and such use forms a further aspect of the invention. Such diseases include inflammatory diseases including asthma, chronic obstructive lung disease, acute adult respiratory distress syndrome, sepsis, rheumatoid and osteo-arthritis, inflammatory bowel disorder, Cröhn's disease and psoriasis, as well as other diseases including myasthenia gravis, schizophrenia, epilepsy, Parkinson's disease, Alzheimer's disease, Tourette's syndrome, chronic pain and nicotine addiction. Patients suffering from such diseases may be administered an effective amount of an agent of the invention. Since many of the above-mentioned conditions are chronic and often incurable, it will be understood that “treatment” is intended to include achieving a reduction in the symptoms for a period of time such as a few hours, days or weeks, and to include slowing the progression of the course of the disease.

Such agents may be formulated into compositions comprising an agent together with a pharmaceutically acceptable carrier or diluent. The agent may in the form of a physiologically functional derivative, such as an ester or a salt, such as an acid addition salt or basic metal salt, or an N or S oxide. Compositions may be formulated for any suitable route and means of administration. Pharmaceutically acceptable carriers or diluents include those used in formulations suitable for oral, rectal, nasal, inhalable, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington=s Pharmaceutical Sciences, Mack Publishing Company, Easton, Pa., 15th Edition, 1975.

The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

This invention will be better understood by reference to the Experimental Details that follow, but those skilled in the art will readily appreciate that these are only illustrative of the invention as described more fully in the claims which follow thereafter. Additionally, throughout this application, various publications are cited. The disclosure of these publications is hereby incorporated by reference into this application to describe more fully the state of the art to which this invention pertains.

### EXPERIMENTAL DETAILS

Cloning of Human α10

The cloning of human α10 is now essentially of historical detail, and is summarised herein to indicate the surprising way in which the clone was achieved. Those of skill in the art may obtain the clone based on the information provided herein based upon the sequences disclosed, as taught in the present application.

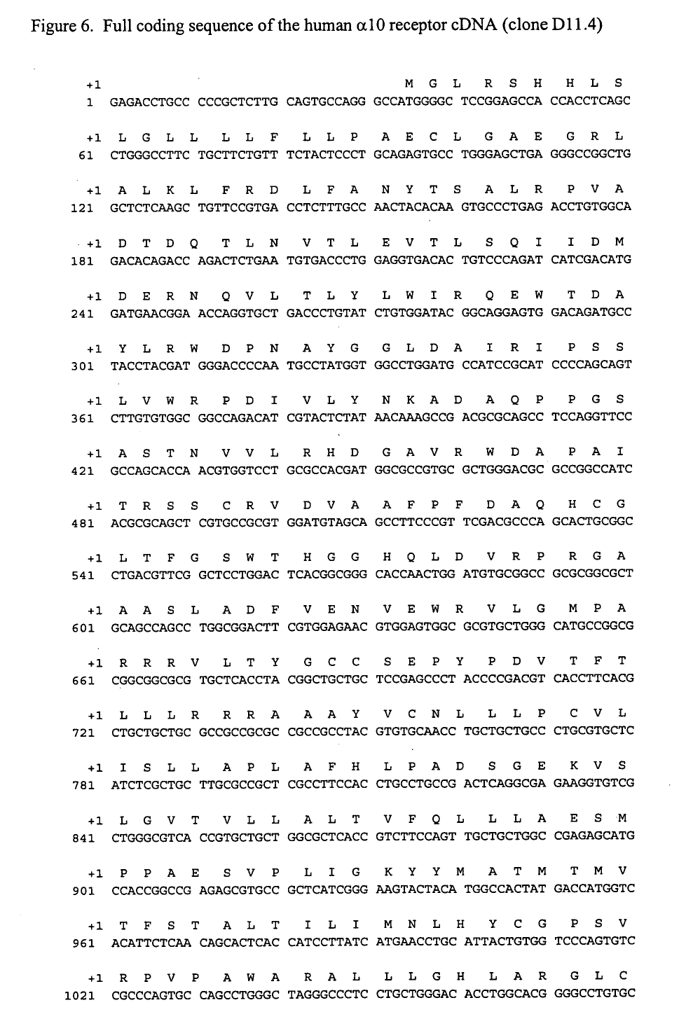

Cloning initially started with the Incyte EST sequence 656293H1 (see FIG. 6 below). This clone was ordered from Incyte, but did not prove to be a full-length sequence and yielded no further information. As other probable human EST “hits” were identified in the public and Incyte databases these were ordered and sequenced, but none were full-length.

The sequence of 656293H1 was used to design a number of oligonucleotide primers for rapid amplification of cDNA ends (RACE) Frohman, M. A. (1991) Methods Enzymol. 218:340-362. Repeated attempts to perform RACE in both 5′ and 3′ directions only extended the sequence by 20 bp in the 3′ direction.

In view of the failure with the RACE experiments, it was decided to attempt to attempt PCR screening of other cDNA (originally developed by Origene) libraries. The sequence of a public domain EST (Genbank accession HSA05001, sequence g1448842 in FIG. 6) was used to design primers for PCR screening of the libraries. A number of positives were identified in an initial screen of pooled cDNAs from a lung cDNA library. A second round of screening (pools containing fewer individual cDNAs) also contained positives. However, on transforming the second round pool into bacteria and screening the colonies obtained, no positives were identified. One possible explanation for this is that there is some growth disadvantage affecting the propagation of α10 cDNA clones in E. coli.

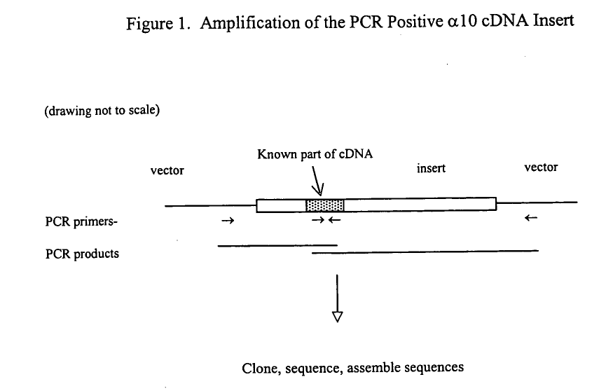

After further research, it was eventually found to be possible to amplify and clone the insert from the cDNA pool in two parts (FIG. 1). Initial DNA sequencing results were disappointing, as the known sequence was only extended slightly. However, after further sequencing it was realised that the clone was a partially-spliced cDNA and could be used for cloning of the spliced cDNA by PCR. The full sequence assembled from the 2 clones as determined in-house is given in FIG. 2.

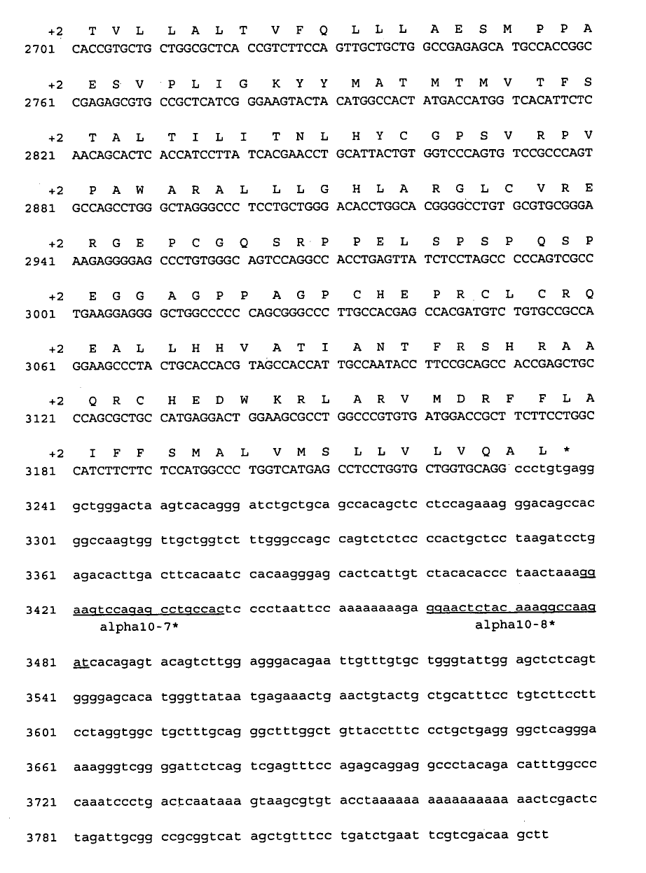

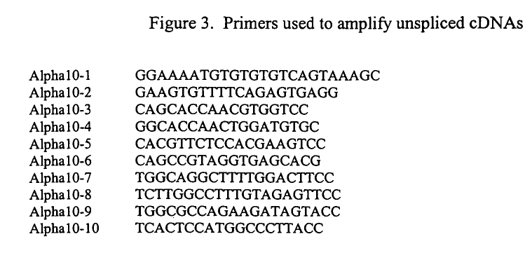

As translation of the most upstream region of homology in the initial clone did not give anything resembling a signal sequence, it was inferred that there would be another upstream intron, and that the translation start had not yet been identified. Therefore PCR primers were designed at the 3′ limit of the regions homologous to the rat and at several points upstream of the recognisable coding sequence (primer positions are shown underlined in FIG. 2, primer sequences are given in FIG. 3). These primers were used to amplify cDNA from pituitary and lymphocyte cDNA libraries (Clontech Marathon-Ready pituitary cDNA, Clontech Marathon-Ready Burkitt's lymphoma cDNA). As expected, a range of product sizes was obtained from both cDNA sources, presumably corresponding to partially spliced mRNAs. These were cloned and sequenced. Several clones appeared to represent spliced cDNAs; by comparing these a consensus sequence could be generated. Said sequence covered the expected mature coding sequence but lacked a signal sequence.

To clone a cDNA covering the full coding sequence, 5=RACE experiments were carried out using a spleen cDNA library (Marathon-ready™ human spleen cDNA, Clontech, Palo Alto Calif. USA). RACE was carried out according to the protocol of Ausubel et al, ibid, using the manufacturer=s primer RACE AP1 and primer NA10 ap2 (CCTCCAGGGTCACATTCAGAGTCTG) followed by amplification using RACE AP1 and NA10 ap3 (CAGCTTGAGAGCCAGCCGGC). RACE products were cloned and sequenced. Based on the sequence of the RACE products, the full-length coding sequence was amplified directly from cerebellum cDNA (Marathon-ready™ human spleen cDNA, Clontech, Palo Alto Calif. USA) using the primers NA10 sp2 (GCGAATTCAGGCCTCACATCCAGAGACCTGC) and Na10 ap8 (CGTCTAGATGACTTAGTCCCAGCCCTCACAGG). PCR products were cloned and sequenced: the sequence of a representative clone is shown in FIG. 6.

This clone has been deposited on 21 Jan. 2000 at the Belgian Coordinated Collection of Microorganisms under the name alpha10/pcDNA3.1/V5 HisA clone D11.4, under the Budapest treaty.

An alignment of the rat α9, the human α9 and the novel human α10, together with the rat α10 and chick α10 polypeptide sequences is shown in FIG. 4. The sequence identities have been calculated based on a 5-way alignment, scoring identity rather than similarity, as follows:

Rat 9 vs human 9 91%

Rat 10 vs human 10 91%

Chick 10 vs human 10 64%

Rat 9 vs human 10 56%

Human 9 vs human 10 56%

For this Pileup was used (Wisconsin Package Version 10.0, Genetics Computer Group (GCG), Madison, Wisc. USA) rather than BLAST, using default parameters.

Expression of Human α10

There is a region of homology to the rat α9 which extends to the N-terminus of the mature rat protein. A corresponding region of the human cDNA is fused to a signal sequence from another protein, Igκ. Accordingly a construct is made in the vector pSecTag2 (Invitrogen), in which the mature human coding sequence encoding SEQ ID NO:2 is fused to an Igκ signal sequence. This construct is tested for ion channel function by methods analogous to those disclosed by Elgoyhen et al, ibid.

The coding sequence of SEQ ID NO:3 was cloned into the EpiTag vector pcDNA3.1/V5 HisA (from Invitrogen), according to the manufacturers instructions. This construct is tested for channel function by standard methods (Elgoyhen et al ibid).

The full coding sequence of the polynucleotides of the invention or the polynucleotide encoding the human alpha 9 subunit (EMBL accession No: AJ243342) have also been transferred into vectors suitable for testing channel function in Xenopus laevis oocytes. Xenopus laevis females were purchased from Blades Biological, UK. The animals were maintained and dissected as described in Gould (1994) Membrane Protein and Expression Systems: a User's Guide Portland Press, London. Stage V or VI oocytes were de-folliculated by incubating with 0.2% collagenase (Sigma) in calcium-free Barth's solution on a vibrating platform set at 8 Hz for 2 hours at 18° C. Oocytes were then maintained in normal Barth's solution (containing 160 IU/ml penicillin and 74 IU/ml streptomycin). On the following day oocytes were injected with 1 to 25 ng cRNA using a Drummond Nanoject and left for at least three days before recording. cRNA was synthesised from cDNAs previously subcloned into the pSP64T.GL+ vector using the SP6 Message Machine Kit (Ambion).

Two electrode voltage clamp recording was carried out using a TURBO TEC-10CD (NPI Electronic GmbH, D-71732 Tamm, Germany). For current recording oocytes were placed in ND-96 Ringer (containing 96 mM NaCl, 1.8 mM CaCl₂, 2 mM KCl, 1 mM MgCl₂ and 5 mM HEPES (pH=7.4)) and impaled with two electrodes filled with 3 M KCl and 1 to 2 MΩ tip resistance. Recording were made in oocytes in which the membrane potential stabilised to potentials more negative than −20 mV during a 20 minute stabilisation period. The membrane potential was clamped at −50 mV and oocytes were continuously perfused with ND96 with or without ACh. In some experiments oocytes were pre-incubated with α-bungarotoxin (α-Btx) prior to application of ACh.

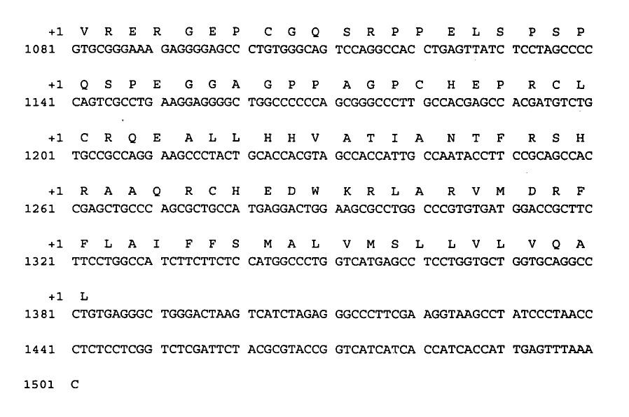

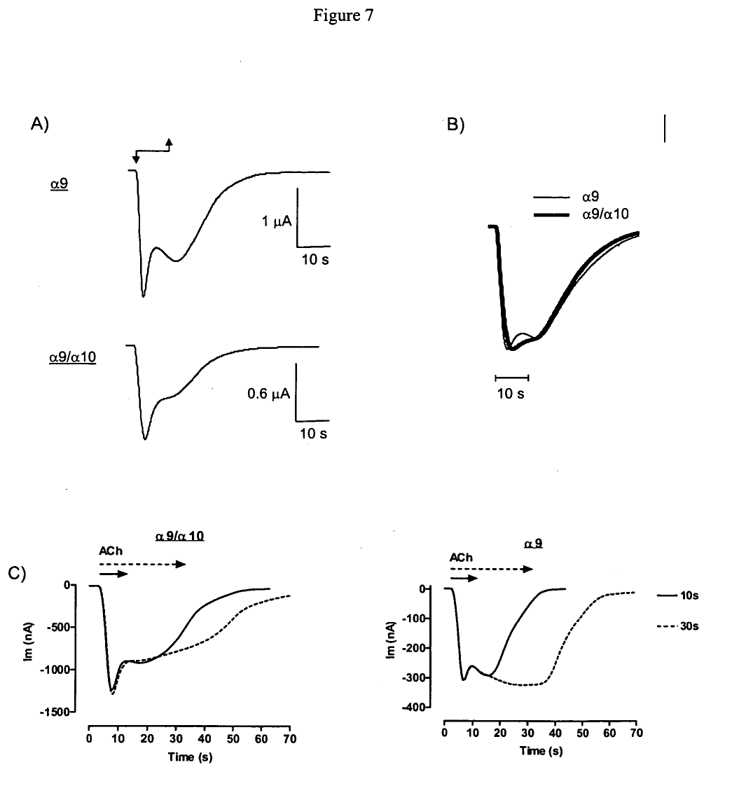

α10 nAChR function and pharmacology was investigated in Xenopus oocytes using two electrode voltage clamp techniques. The neurotransmitter acetylcholine (ACh) induced inward currents with a complex time course of decay in oocytes into which both α10 and α9 subunits had been injected concurrently which were distinct from currents observed in oocytes injected with α9 alone. FIG. 7A shows that the ACh-induced current in an α9/α10 injected oocyte is biphasic, activating to reach a peak and then decaying rapidly to a plateau after which the current decays more slowly towards the baseline. In contrast, the ACh-induced current in an α9 injected oocyte activates and decays rapidly but then reactivates after washout of the ACh before decaying to the baseline. The means of 16 to 19 currents from each type of oocyte normalised to the first peak current (FIG. 7B) reveals that the α9/α10 combination activates slower and decays faster than the α9. The re-activation of the current observed in α9 is sustained by prolonged application (e.g. 30 s) of ACh (FIG. 7C) and the current only decays after removal of the ACh (observed in two oocytes). For the α9/α10 combination there is no re-activation of current with a 30 s ACh application, although the duration of the decaying current is extended beyond the time observed for a 10 s application of ACh (observed in two oocytes). Thus compared to the responses evoked by a 10 s ACh application the proportional increase in area-under-the curve for normalised responses evoked by a 30 s ACh application for the α9/α10 (44%; N=2) combination is less than for α9 alone (82%; N=2).

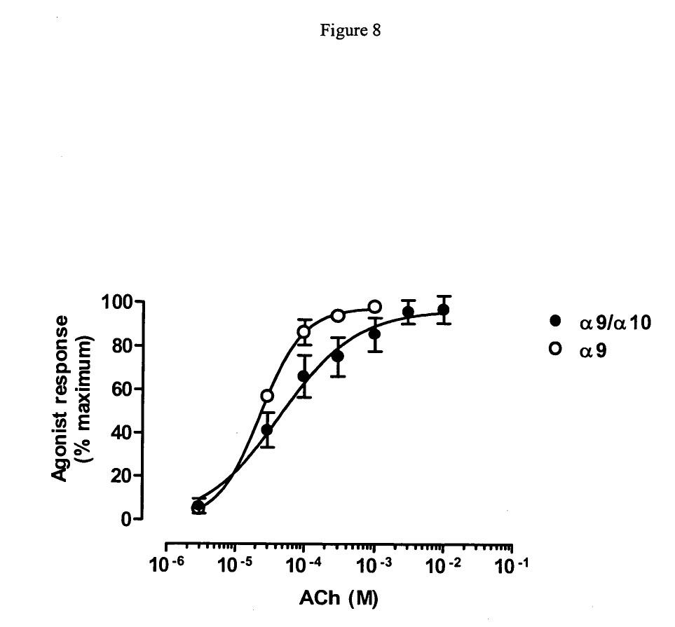

Comparison of the concentration-response curves to ACh (FIG. 8) shows that the α9/α10 combination is less sensitive to ACh than α9 alone. Thus the EC50 for activation of nicotinic current in α9/α10 (46 μM) is half that of α9 (24 μM). In addition, the Hill coefficient for the curve is less for α9/α10 (0.81) compared to α9 (1.41). This is consistent with a lower co-operativity of α9/α10 activation compared to α9 activation by ACh and can be explained by there being fewer binding sites for ACh in α9/α10 combination than in the homomeric α9.

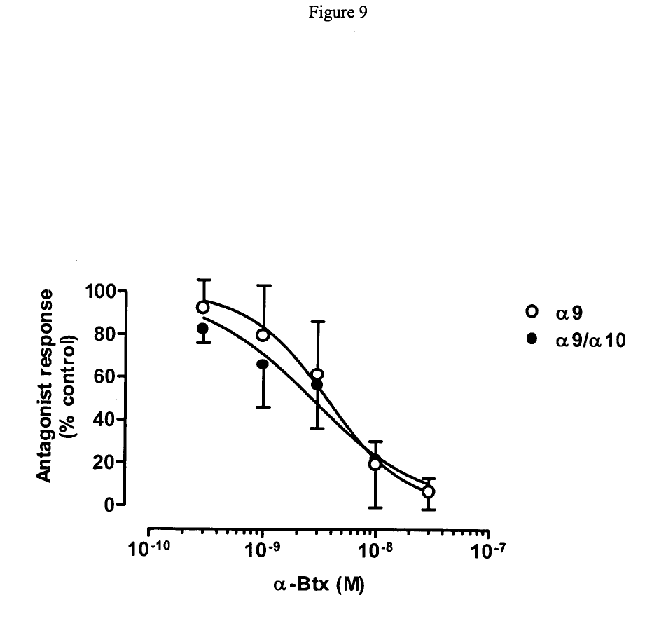

α9 nAChRs are highly sensitive to the snake toxin α-Btx. Therefore the action of this inhibitor on standard nicotinic responses (evoked by ACh concentrations at the EC50 for either the α9/α10 combination or α9 alone) was compared in oocytes expressing either α9/α10 or α9 alone. Comparison of the concentration-response curves to α-Btx (FIG. 9) show that slope of the inhibition of ACh evoked response was shallower in the α9/α10 combination compared to the α9 alone. The lower Hill coefficient for the α9/α10 combination inhibition curve (−0.89) versus α9 alone (−1.25) is consistent with the observations on ACh concentrations-response curves mentioned above. α-Btx potently inhibits both the α9/α10 combination and α9 alone with IC50s of 3.7 and 2.7 nM, respectively.

mRNA Distribution

Human α10 mRNA has been successfully amplified by nested PCR (for protocol see Ausubel et al, ibid) from a number of different sources as indicated in Table 1.

TABLE 1

| | |
|---|---|
| Pituitary Gland | Clontech Marathon-Ready Pituitary cDNA* |
| Lymphoma | Clontech Marathon-Ready Burkit's cDNA (Raji)* |
| Liver | Contech Quick-Screen cDNA library |
| Peripheral blood | Clontech Quick-Clone cDNA |
| leukocyte | |
| Normal tongue | Invitrogen Gene Pool normal tongue (special order) |
| Lymphoma | Contech 5′ stretch cDNA library (Burkitt's, |
| | Daudi) |
| Testis | Clontech Marathon-Ready cDNA |
| Spleen | Clontech Marathon-Ready cDNA |

*full-length fragments have been amplified and cloned from these sources.

α10 Localisation in Rat Brain by In Situ Hybridization

Experiments were performed according to a protocol described in Bonaventure et al., (1998, Neuroscience 82: 469-484) using 20 μm sagittal sections of adult Wistar rat brains. ³³P RNA probes were generated from an α10 fragment encoding nucleotides 7-382 of the cDNA. Antisense and sense (as negative control) riboprobes were hybridized overnight at 40° C. Post-hybridization washes were performed at 50° C. Dried sections were apposed to phosphorimager plates for 1 week. Plates were read (Fuji BAS 2500) and converted into digitized images. Antisense riboprobes to rat α10 gave specific hybridisation signals in the white matter of the cerebellum or in discrete areas of the hypothalamo-pituitary axis compared to the control sense probe. The long exposures necessary to reveal these signals suggests that the expression of α10nAChR subunit mRNA was very low.

Chromosomal Mapping of α10

The BLASTN program (Altschul et al (1997) Nucleic Acids Res. 25 3389-3402) was used to map the α10 cDNA onto the draft of the human genome sequence (ensemble database EMBL:www.ebi.ac.uk). α10 co-localised with NUP98 (accession no U41815) which has been mapped previously to chromosome 11 p15.5 (Borrow et al, 1996, Nature Genetics 14, 33-41). The interval containing the α10 and NUP98 sequences is flanked by reference markers D11S1318 and D11S909. This region spans approximately 6-16 centimorgans and resides on radiation hybrid RHdb RH18062. The physical position is indicated as 39.58 cR3000 (P0.29). The locus has previously been implicated by genetic linkage studies with psychiatric disorders such as schizophrenia (Coon et al, 1993, Biol. Psychiatry 34 277-289).

1. (canceled)

2. An isolated nucleic acid sequence comprising SEQ ID NO:1.

3. A recombinant DNA molecule comprising SEQ ID NO:1.

4. (canceled)

5. (canceled)

6. (canceled)

7. (canceled)

8. An isolated nucleic acid encoding a polypeptide, said isolated nucleic acid comprising SEQ ID NO:1.

9. An expression vector comprising a nucleic acid as defined in claim 8 operably linked to a promoter.

10. A host cell carrying a vector according to claim 9.

11. (canceled)

12. (canceled)

13. (canceled)

14. (canceled)

15. (canceled)

16. (canceled)

17. (canceled)

18. (canceled)

19. The DNA molecule of claim 8 wherein the polypeptide has a sequence corresponding to SEQ ID NO:2.

20. A pair of nucleic acid molecules, comprising a first nucleic acid consisting essentially of a fragment of at least 15 nucleotides capable of hybridizing to SEQ ID NO:1 and a second nucleic acid consisting essentially of a fragment of at least 15 nucleotides capable of hybridizing to the complement of SEQ ID NO: 1, wherein the pair is capable of directing the amplification of at least a portion of SEQ ID NO:1 in a polymerase chain reaction.
